(19) United States
(12) Patent Application Publication
(10) Pub. No.: US 2024/0199577 A1
(43) Pub. Date: Jun. 20, 2024

(54) ACORAMIDIS ANALOGS FOR STABILIZING TRANSTHYRETIN AND INHIBITING TRANSTHYRETIN MISFOLDING

(71) Applicant: Protego Biopharma, Inc., San Diego, CA (US)

(72) Inventors: Richard F. Labaudiniere, Charleston, SC (US); Bradley Dean Tait, North Andover, MA (US)

(21) Appl. No.: 18/419,303

(22) Filed: Jan. 22, 2024

Related U.S. Application Data
(63) Continuation of application No. PCT/US2022/038506, filed on Jul. 27, 2022.
(60) Provisional application No. 63/203,685, filed on Jul. 28, 2021.

(51) Int. Cl. C07D 401/12; A61K 31/4155; A61K 31/4178; A61K 31/4439; C07D 231/12; C07D 403/12

(52) U.S. Cl. CPC C07D 401/12; A61K 31/4155; A61K 31/4178; A61K 31/4439; C07D 231/12; C07D 403/12

(57) ABSTRACT

Provided herein are compounds having activity against TTR related conditions, and pharmaceutically accepted salts and solvates thereof. Also provided are methods of using the compounds for inhibiting and preventing TTR aggregation and/or amyloid formation in the peripheral nerves, kidney, cardiac tissue, eye and CNS, and of treating a subject with peripheral TTR amyloidosis.

### RELATED APPLICATIONS

This application is a continuation under 35 USC § 120 of International Application No. PCT/US2022/038506, filed Jul. 27, 2022, which claims priority to U.S. provisional application No. 63/203,685, filed Jul. 28, 2021. The contents of the above-referenced applications are incorporated by reference herein in their entirety.

### BACKGROUND

Transthyretin (TTR) amyloidosis is a severely debilitating, and ultimately fatal, systemic condition induced by the accumulation of TTR amyloid within tissues in amounts sufficient to impair normal function. The transthyretin (TTR) amyloidoses (ATTR) are fatal progressive sporadic (WT TTR aggregates) or autosomal dominant degenerative diseases (mutant and WT TTR aggregates). The ATTR's are caused by dissociation of tetramer TTR subunits, followed by monomer misfolding, and misassembly into a spectrum of TTR aggregate structures, including amyloid fibrils. TTR is synthesized and secreted primarily by the liver (which is not a site of aggregate deposition) into the blood, by retinal pigment and ciliary pigment epithelial cells into the eye, and by the choroid plexus into the central nervous system (CNS). The clinical expression is variable among different mutations and different populations, and even the same population with the same mutation can present significant variability. The age of onset varies between the 20s and the 90s. The TTR amyloidoses present with a diversity of symptoms and phenotypes, including peripheral polyneuropathy, autonomic neuropathy, cardiomyopathy, carpal tunnel syndrome, ocular amyloid angiopathy and leptomeningeal amyloid angiopathy, reflecting the different sources of TTR synthesis and the susceptibilities of various tissues to discrete toxic aggregate structures comprised of different TTR sequences. The peripheral nerves and the heart are the organs most frequently affected by TTR amyloid deposition, leading to ATTR-familial amyloid polyneuropathy (ATTR-FAP) and ATTR-familial amyloid cardiomyopathy (ATTR-FAC), respectively. Wild-type TTR can also be deposited as amyloid, particularly in the heart leading to wild-type transthyretin amyloid, also known as senile systemic amyloidosis (SSA). The main feature of ATTR-FAP is progressive, length-dependent degenerative sensorimotor and autonomic neuropathy. Cardiac involvement in ATTR can range from asymptomatic atrioventricular block to severe and rapidly progressive cardiomyopathy and heart failure and include arrhythmias and conduction disturbances, and cardiac infiltration with ventricular wall thickness progressing to heart failure. Average life expectancy in symptomatic FAP without treatment is 10 years, in FAC and SSA it is perhaps half that or less. Deposition of TTR amyloid in the eye and brain are associated with oculoleptomeningeal amyloidosis (ATTR-OLMA), a rare form of TTR amyloidosis with an average life expectancy of 4 to 12 years after onset. The sources of misfolded TTR in the brain and eye are the choroid plexus, the retinal pigment epithelium and ciliary pigment epithelium, respectively. TTR oculopathy is characterized, initially by dry eyes, then by progressive TTR amyloid deposition in the iris and anterior capsule of the lens. Conjunctival amyloid vasculopathy, scalloped pupils, glaucoma, vitreous opacities and finally retinal amyloid angiopathy complete the ocular pathological cascade. Vitreous opacity is treated by vitrectomy and intraocular lens implantation, however recurrent vitreous opacities occur in 14% of the treated eyes. Glaucoma is a major ocular manifestation in ATTR patients and the leading cause of irreversible blindness in these patients. Occurrence of glaucoma in this patient population is significantly increased in eyes with amyloid deposition (vitreous opacity, amyloid deposition on the pupils, fringed pupils and scalloped pupils). Trabeculectomy with mitomycin C is a standard eye surgical treatment in moderate and advanced glaucoma patients. The surgical probability of success of trabeculectomy, at 5 years, is very low (<20%) in ATTR patients, compared to 70% in non-TTR glaucoma patients. Post-surgery complications of ocular decompression retinopathy and neovascular glaucoma, caused by amyloid angiopathy are significantly increased in ATTR patient population. In addition, TTR amyloid deposition in the meninges and vessels of the brain and spinal cord is manifested clinically by transient focal neurological episodes (TFNE) most common 10-15 years after disease onset. TFNEs include transient ischemic attack-like episodes, stroke, aura-like episodes and epileptic seizures—with symptoms lasting several min to several hours to days. TFNEs frequency, duration of symptoms and cerebral TTR amyloid deposition increase with time. The phenotype-genotype relationships in ATTR are not completely understood. More than 100 TTR mutations have been associated with ATTR. Historically, several one point mutations have been associated with one major phenotype: V30M for ATTR-PN, V122I and wt for ATTR-FAC, D18G and Y114C for oculoleptomeningeal amyloidosis. In fact, most of the TTR variants are associated with mixed phenotypes. As ATTR is a systemic disease, other organs can become involved as the disease progresses. Recent evidences suggest that ocular and CNS amyloid depositions occur in a large proportion of ATTR-FAP patients and can become manifest 5-15 years post polyneuropathy onset and in those patients with long-standing disease and with extended survival after effective treatment targeting peripheral symptoms. Cerebral imaging by 11C-PiB PET-scan and brain biopsies indicates that cerebral TTR amyloid deposition exists prior to any overt CNS manifestations (10 years before FNE onset). Amyloid deposition is found in conjunctival vessel walls in 89% of V30M TTR-FAP patients prior to vitreous opacity. Depositions of amyloid on iris and anterior capsule of the lens are present in 40% of V30M TTR-FAP patients at 15 years post disease onset, in 70% at 20 years and above 80% at 25 yrs. Since 1993, liver transplantation (LT), in which the liver producing the amyloidogenic mutant TTR protein is replaced by one producing wild-type TTR, a crude form of gene therapy, was the only treatment option for ATTR-FAP. The 10-year patient survival is 79% in patients with the V30M TTR variant after LT. Clinical improvement of sensory neuropathy has been observed in 42% of subjects during the first 6 months after LT. However, LT does not prevent locally synthesized mutant-TTR amyloid deposition in the eye and brain. Variant TTR amyloid deposition has been found in vitreous humor and brains of LT ATTR-FAP patients. With or without LT treatment, prevalence of all ocular manifestations increases with disease duration. Glaucoma and vitreous opacity prevalence is up to 25% at 25 yrs. In fact, a significantly higher prevalence of amyloid deposition on the iris, on the anterior capsule of the lens and in the vitreous, and of scalloped iris is observed in liver transplanted patients versus non-transplanted patients. Furthermore, up to 31% of post-LT V30M ATTR-FAP patients will develop focal CNS manifestations 10 to 15 years post disease onset. The frequency of both cerebral amyloid deposition and FNE's increase with disease duration post LT. Tafamidis, a small molecule TTR stabilizer that inhibits TTR dissociation, misfolding and aggregation has been approved for the treatment of ATTR-FAP in the US, EU, Japan and Brazil and in 37 additional countries. The drug is well tolerated and treatment is associated with a significant delay in the progression of peripheral neurological impairment. Tafamidis treatment significantly increase survival when compared to the natural course of the disease. In a survey conducted examining clinical data from 11 sites (in 6 countries), V30M ATTR patients treated with tafamidis or LT continue to develop ocular symptoms, vitreous opacity and glaucoma. Moreover, tafamidis failed to halt progression of oculoleptomeningeal amyloidosis in a Ala36Pro TTR patient. Tafamidis brain and eye penetrance is not sufficient to stop TTR aggregation in the eye and CNS. Despite the much lower TTR concentration in CSF and the eye compared to that in plasma (0.4-2.8 mg/dL in CSF, 0.6 mg/dL in eye versus 16-35 mg/dL in plasma), tafamidis levels in CSF and vitreous of currently tafamidis-treated FAP patients are only 2% and 0.5%, respectively, of that in plasma, leading to low tafamidis/TTR stoechimetric ratio: ≤1 in vitreous and CSF versus 2.4 in plasma. Similarly, while promising in the treatment of peripheral disease, siRNA (Alnylam) and ASO's (Ions) directed against TTR, as currently formulated, are unable to penetrate the eye or the brain, rendering them ineffective in treating the cerebral and ocular components of the TTR amyloidoses. Further, AG10 (acoramidis), a TTR stabilizer currently in clinical evaluation, has poor brain penetration. Thus, even with the considerable progress made in therapeutic management of ATTR-FAP, the ocular and CNS manifestations of ATTR represent a significant unmet medical need, especially when considering the prospect of prolonged survival of such patients with current treatments or those under development that effectively halt peripheral disease progression. It is probable that, with prolonged survival, serious eye disease and CNS manifestations may occur in a large proportion of ATTR patients.

### SUMMARY

Provided herein are compounds, compositions and methods for stabilizing transthyretin misfolding. In one embodiment, the compounds for use in the compositions and methods provided herein have Formula I. In another embodiment, the compounds for use in the compositions and methods provided herein have Formula II.

Also provided herein are methods of treatment of diseases and disorders resulting from transthyretin misfolding by administering a compound or composition provided herein. Further provided are methods of treatment of diseases or disorders resulting from transthyretin amyloidosis by administering a compound or composition provided herein. In other embodiments, provided herein is a method of inhibiting and preventing transthyretin aggregation and/or amyloid formation in the eye or CNS by administering a compound or composition provided herein. In another embodiment, provided herein is a method of treatment of peripheral transthyretin amyloidosis or ocular or cerebral amyloid angiopathy by administering a compound or a composition provided herein. In other embodiments, provided herein is a method of treatment of familial amyloid polyneuropathy, familial amyloid cardiomyopathy, localized nodular cutaneous amyloidosis, TTR oculoleptomeningeal amyloidosis or senile systemic amyloidosis by administering a compound or a composition provided herein.

### DETAILED DESCRIPTION

### I. Definitions

The abbreviations used herein have their conventional meaning within the chemical and biological arts.

Where moieties are specified by their conventional chemical formulae, written from left to right, they equally encompass the chemically identical moieties that would result from writing the structure from right to left, e.g., —CH₂O— is equivalent to —OCH₂—.

The term “alkyl,” by itself or as part of another substituent, means, unless otherwise stated, a straight (i.e., unbranched) or branched chain saturated hydrocarbon radical, which can include di- and multivalent radicals, having the number of carbon atoms designated (i.e. C₁-C₁₀ means one to ten carbons). Examples of alkyl groups include, but are not limited to, groups such as methyl, ethyl, n-propyl, isopropyl, n-butyl, t-butyl, isobutyl, sec-butyl, homologs and isomers of, for example, n-pentyl, n-hexyl, n-heptyl, n-octyl, and the like.

The term “alkenyl,” by itself or as part of another substituent, means, unless otherwise stated, a straight (i.e., unbranched) or branched chain hydrocarbon radical having one or more carbon-carbon double bonds, which can include di- and multivalent radicals, having the number of carbon atoms designated (i.e., C₁-C₁₀ means one to ten carbons). Examples of alkenyl groups include, but are not limited to, vinyl (i.e., ethenyl), 2-propenyl, crotyl, 2-isopentenyl, 2-(butadienyl), 2,4-pentadienyl, 3-(1,4-pentadienyl), and the higher homologs and isomers.

The term “alkynyl,” by itself or as part of another substituent, means, unless otherwise stated, a straight (i.e., unbranched) or branched chain hydrocarbon radical having one or more carbon-carbon triple bonds, which can include di- and multivalent radicals, having the number of carbon atoms designated (i.e. C₁-C₁₀ means one to ten carbons). Examples of alkynyl groups include, but are not limited to, ethynyl, 1- and 3-propynyl, 3-butynyl, and the higher homologs and isomers.

The term “alkylene” by itself or as part of another substituent means a divalent radical derived from an alkyl, as exemplified, but not limited, by —CH₂CH₂CH₂CH₂—. Typically, an alkyl (or alkylene) group will have from 1 to 24 carbon atoms, including those groups having 10 or fewer carbon atoms. A “lower alkyl” or “lower alkylene” is a shorter chain alkyl or alkylene group, generally having eight or fewer carbon atoms.

The terms “alkoxy,” “alkylamino,” and “alkylthio” (or thioalkoxy) are used in their conventional sense, and refer to those alkyl groups attached to the remainder of the molecule via an oxygen atom, an amino group, or a sulfur atom, respectively.

The term “heteroalkyl,” by itself or in combination with another term, means, unless otherwise stated, a stable straight or branched chain, or cyclic hydrocarbon radical, or combinations thereof, consisting of the stated number of carbon atoms and a heteroatom selected from the group consisting of O, N, P, Si and S, and wherein the nitrogen and sulfur atoms may optionally be oxidized and the nitrogen heteroatom may have an alkyl substituent to fulfill valency and/or may optionally be quaternized. The heteroatom(s) O, N, P and S and Si may be placed at any interior position of the heteroalkyl group or at the position at which the alkyl group is attached to the remainder of the molecule. Examples include, but are not limited to, —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CH—O—CH₃, —Si(CH₃)₃, —CH₂—CH═N—OCH₃, —CH═CH—N(CH₃)—CH₃, O—CH₃, —O—CH₂—CH₃, and —CN. Up to two heteroatoms may be consecutive, such as, for example, —CH₂—NH—OCH₃ and —CH₂—O—Si(CH₃)₃. Similarly, the term “heteroalkylene” by itself or as part of another substituent means a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′— and —R′C(O)₂—. As described above, heteroalkyl groups, as used herein, include those groups that are attached to the remainder of the molecule through a heteroatom, such as —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like.

The terms “cycloalkyl” and “heterocycloalkyl”, by themselves or in combination with other terms, represent, unless otherwise stated, cyclic versions of “alkyl” and “heteroalkyl”, respectively, including bicyclic, tricyclic and bridged bicyclic groups. Additionally, for heterocycloalkyl, a heteroatom can occupy the position at which the heterocycle is attached to the remainder of the molecule. Examples of cycloalkyl include, but are not limited to, cyclopentyl, cyclohexyl, 1-cyclohexenyl, 3-cyclohexenyl, cycloheptyl, norbornanyl, bicyclo[2.2.2]octanyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, 1- or 2-azabicyclo[2.2.2]octanyl, and the like.

The terms “halo,” by itself or as part of another substituent, means, unless otherwise stated, a fluorine, chlorine, bromine, or iodine atom. Additionally, terms such as “haloalkyl,” are meant to include monohaloalkyl and polyhaloalkyl. For example, the term “halo(C₁-C₄)alkyl” is meant to include, but not be limited to, trifluoromethyl, 2,2,2-trifluoroethyl, 4-chlorobutyl, 3-bromopropyl, and the like.

The term “aryl” means, unless otherwise stated, a polyunsaturated, aromatic, hydrocarbon substituent which can be a single ring or multiple rings (in one embodiment from 1 to 3 rings) which are fused together or linked covalently. The term “heteroaryl” refers to aryl groups that contain from one to four heteroatoms selected from N, O, and S in the ring(s), wherein the nitrogen and sulfur atoms are optionally oxidized, and the nitrogen atom(s) are optionally quaternized. A heteroaryl group can be attached to the remainder of the molecule through a carbon or heteroatom. Non-limiting examples of aryl and heteroaryl groups include phenyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituent moieties for aryl and heteroaryl ring systems may be selected from the group of acceptable substituent moieties described herein. The term “heteroarylium” refers to a heteroaryl group that is positively charged on one or more of the heteroatoms.

The term “oxo” as used herein means an oxygen atom that is double bonded to a carbon atom.

Each of the above terms (e.g., “alkyl,” “heteroalkyl,” “aryl” and “heteroaryl”) are meant to include both substituted and unsubstituted forms of the indicated radical. Non-limiting examples of substituent moieties for each type of radical are provided below.

Substituent moieties for alkyl, heteroalkyl, alkylene, alkenyl, heteroalkylene, heteroalkenyl, alkynyl, cycloalkyl, heterocycloalkyl, cycloalkenyl, and heterocycloalkenyl groups are, in one embodiment, selected from, —OR′, ═O, ═NR′, ═N—OR′, —NR′R″, —SR′, halo, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R′″)═NR″″, —NR—C(NR′R″)═NR′″, —S(O)R′, —S(O)₂R′, —S(O)₂NR′R″, —NRSO₂R′, —CN and —NO₂ in a number ranging from zero to the number of hydrogen atoms in such radical. R′, R″, R″′ and R″″ each in one embodiment independently are hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted alkyl, alkoxy or thioalkoxy groups, or arylalkyl groups. When a compound provided herein includes more than one R group, for example, each of the R groups is independently selected as are each R′, R″, R′″ and R″″ groups when more than one of these groups is present. When R′ and R″ are attached to the same nitrogen atom, they can be combined with the nitrogen atom to form a 4-, 5-, 6-, or 7-membered ring. For example, —NR′R″ is meant to include, but not be limited to, 1-pyrrolidinyl and 4-morpholinyl. From the above discussion of substituent moieties, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Substituent moieties for aryl and heteroaryl groups are, in one embodiment, selected from halo, —OR′, —NR′R″, —SR′, —SiR′R″R″′, —OC(O)R′, —C(O)R′, —CO₂R′, —CONR′R″, —OC(O)NR′R″, —NR″C(O)R′, —NR′—C(O)NR″R″′, —NR″C(O)₂R′, —NR—C(NR′R″R′″)═NR″, —NR—C(NR′R″)═NR′″, —S(O)R′, —S(O)₂R′, —S(O)₂NR′R″, —NRSO₂R′, —CN and —NO₂, —R′, —N₃, —CH(Ph)₂, fluoro(C₁-C₄)alkoxy, and fluoro(C₁-C₄)alkyl, in a number ranging from zero to the total number of hydrogens on the aromatic ring system; and where R′, R″, R″′ and R″″ are, in one embodiment, independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl and substituted or unsubstituted heteroaryl. When a compound provided herein includes more than one R group, for example, each of the R groups is independently selected as are each R′, R″, R″′ and R″″ groups when more than one of these groups is present.

Two of the substituent moieties on adjacent atoms of an aryl or heteroaryl ring may optionally form a ring of the formula -Q′-C(O)—(CRR′)_{q}-Q″-, wherein Q′ and Q″ are independently —NR—, —O—, —CRR′— or a single bond, and q is an integer of from 0 to 3. Alternatively, two of the substituent moieties on adjacent atoms of the aryl or heteroaryl ring may optionally be replaced with a substituent of the formula -A-(CH₂)ᵣ—B—, wherein A and B are independently —CRR′—, —O—, —NR—, —S—, —S(O)—, —S(O)₂—, —S(O)₂NR′— or a single bond, and r is an integer of from 1 to 4. One of the single bonds of the new ring so formed may optionally be replaced with a double bond. Alternatively, two of the substituent moieties on adjacent atoms of the aryl or heteroaryl ring may optionally be replaced with a substituent of the formula —(CRR′)ₛ—X′—(C″R″′)_{d}—, where s and d are independently integers of from 0 to 3, and X′ is —O—, —NR′—, —S—, —S(O)—, —S(O)₂—, or —S(O)₂NR′—. The substituent moieties R, R′, R″ and R″′ are, in one embodiment, independently selected from hydrogen, substituted or unsubstituted alkyl, substituted or unsubstituted cycloalkyl, substituted or unsubstituted heterocycloalkyl, substituted or unsubstituted aryl, and substituted or unsubstituted heteroaryl.

As used herein, the term “heteroatom” or “ring heteroatom” is meant to include oxygen (O), nitrogen (N), sulfur (S), phosphorus (P), and silicon (Si).

The term “pharmaceutically acceptable salts” refers to salts of the compounds provided herein which are prepared with relatively nontoxic acids or bases known to those of skill in the art, depending on the particular substituent moieties found on the compounds provided herein. When compounds provided herein contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds provided herein contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids such as hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge et al., “Pharmaceutical Salts”, Journal of Pharmaceutical Science, 1977, 66, 1-19). Certain compounds provided herein contain both basic and acidic functionalities that allow the compounds to be converted into either base or acid addition salts.

The neutral forms of the compounds provided herein are in one embodiment regenerated by contacting the salt with a base or acid and isolating the parent compound in a conventional manner known to those of skill in the art.

As used herein, a prodrug is a compound that upon in vivo administration is metabolized, or otherwise undergoes chemical changes under physiological conditions, by one or more steps or processes or otherwise converted to a biologically, pharmaceutically or therapeutically active form of the compound. Additionally, prodrugs can be converted to a biologically, pharmaceutically or therapeutically active form of the compound by chemical or biochemical methods in an ex vivo environment. For example, prodrugs can be converted to the compounds of the present invention when placed in a transdermal patch reservoir with a suitable enzyme or chemical reagent.

Certain compounds provided herein can exist in unsolvated forms as well as solvated forms, including hydrated forms. In general, the solvated forms are equivalent to unsolvated forms and are encompassed within the scope of the present disclosure. Certain compounds provided herein may exist in multiple crystalline or amorphous forms. In general, all physical forms are equivalent for the uses contemplated herein and are intended to be within the scope of the present disclosure.

Certain compounds provided herein possess asymmetric carbon atoms (optical centers) or double bonds; the racemates, diastereomers, tautomers, geometric isomers and individual isomers are encompassed within the scope of the present disclosure. The compounds provided herein do not include those which are known in the art to be too unstable to synthesize and/or isolate.

The compounds provided herein may also contain unnatural proportions of atomic isotopes at one or more of the atoms that constitute such compounds. For example, the compounds may be radiolabeled with radioactive isotopes, such as for example tritium (³H), iodine-125 (¹²⁵I) or carbon-14 (¹⁴C). All isotopic variations of the compounds provided herein, whether radioactive or not, are encompassed within the scope of the present disclosure.

In some embodiments, each substituted aryl and/or heterocycloalkyl is substituted with a substituent group, a size limited substituent group, or a lower substituent group. A “substituent group,” as used herein, means a group selected from the following moieties:

—OH, —NH₂, —SH, —CN, —CF₃, oxo, halo, unsubstituted alkyl, unsubstituted heteroalkyl, unsubstituted cycloalkyl, unsubstituted heterocycloalkyl, unsubstituted aryl, unsubstituted heteroaryl, and

alkyl, heteroalkyl, cycloalkyl, heterocycloalkyl, aryl, and heteroaryl, substituted with at least one substituent selected from:
(i) oxo, —OH, —NH₂, —SH, —CN, —CF₃, halo, unsubstituted alkyl, unsubstituted heteroalkyl, unsubstituted cycloalkyl, unsubstituted heterocycloalkyl, unsubstituted aryl, unsubstituted heteroaryl, and
(ii) alkyl, heteroalkyl, cycloalkyl, heterocycloalkyl, aryl, and heteroaryl, substituted with at least one substituent selected from:
   (a) oxo, —OH, —NH₂, —SH, —CN, —CF₃, halo, unsubstituted alkyl, unsubstituted heteroalkyl, unsubstituted cycloalkyl, unsubstituted heterocycloalkyl, unsubstituted aryl, unsubstituted heteroaryl, and
   (b) alkyl, heteroalkyl, cycloalkyl, heterocycloalkyl, aryl, or heteroaryl, substituted with at least one substituent selected from oxo, —OH, —NH₂, —SH, —CN, —CF₃, halo, unsubstituted alkyl, unsubstituted heteroalkyl, unsubstituted cycloalkyl, unsubstituted heterocycloalkyl, unsubstituted aryl, and unsubstituted heteroaryl.

A “size-limited substituent” or “ size-limited substituent group,” as used herein means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₄-C₈ cycloalkyl, and each substituted or unsubstituted heterocycloalkyl is a substituted or unsubstituted 4 to 8 membered heterocycloalkyl.

A “lower substituent” or “ lower substituent group,” as used herein means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₅-C₇ cycloalkyl, and each substituted or unsubstituted heterocycloalkyl is a substituted or unsubstituted 5 to 7 membered heterocycloalkyl.

The term “treating” refers to any indicia of success in the therapy or amelioration of one or more symptoms of an injury, pathology or condition, including any objective or subjective parameter such as abatement; remission; diminishing of symptoms or making the injury, pathology or condition more tolerable to the patient; slowing in the rate of degeneration or decline; making the final point of degeneration less debilitating; improving a patient's physical or mental well-being. The therapy or amelioration of symptoms can be based on objective or subjective parameters; including the results of a physical examination, neuropsychiatric exams, and/or a psychiatric evaluation. For example, in one embodiment, the methods provided herein successfully treat a patient's delirium by decreasing the incidence of disturbances in consciousness or cognition.

Solid and dashed wedge bonds indicate stereochemistry as customary in the art. A “squiggle” bond (i.e., “ [symbol] ” indicates either R- or S-stereochemistry.

### II. Compounds and Compositions

In one embodiment, provided herein is a compound for use in the compositions and methods provided herein having the structure of Formula I, or pharmaceutically acceptable salt or solvate thereof:

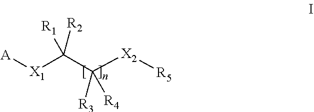

wherein:

A is

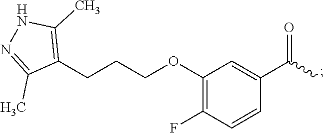

X₁ is O, S or NR₆;

X₂ is a bond, O, S, NR₆, N⁺R₆R₇ or P⁺(Ar)₂;

X₃ is O, S or NR₆;

n is an integer from 0-6;

m is an integer from 0-6;

Ar is aryl, heteroaryl or heteroarylium (all optionally substituted);

R₁, R₂, R₃, R₄, R₅, R₆ and R₇ are each independently H, halo, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OH, OR₈, COR₉, COOR₉, —(CR₁₀R₁₁)ₘX₃R₈, or —(CR₁₀R₁₁)ₘX₃COR₉;

R₈ and R₉ are each independently H, alkyl, haloalkyl, cycloalkyl, heterocycloalkyl, alkenyl, cycloalkenyl, heterocycloalkenyl, aryl, heteroaryl, aralkyl or heteroaralkyl (all optionally substituted);

or R₈ is selected as above and R₉ is:

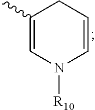

and

R₁₀ and R₁₁ are each independently H, alkyl, haloalkyl, cycloakyl, heterocycloalkyl, alkenyl, cycloalkenyl, heterocycloalkenyl, aryl, heteroaryl, aralkyl, heteroaralkyl (all optionally substituted) or OR₈.

In another embodiment, the compounds provided herein have Formula I, or pharmaceutically acceptable salt or solvate thereof, wherein:
A is

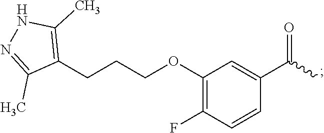

X₁ is O, S or NR₆;

X₂ is a bond, O, S, NR₆, N⁺R₆R₇ or P⁺(Ar)₂;

X₃ is O, S or NR₆;

n is an integer from 0-6;

m is an integer from 0-6;

Ar is aryl, heteroaryl or heteroarylium (all optionally substituted);

R₁, R₂, R₃, R₄, R₅, R₆ and R₇ are each independently H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OH, OR₈, COR₉, —(CR₁₀R₁₁)ₘX₃R₈, or —(CR₁₀R₁₁)ₘX₃COR₉;

R₈ and R₉ are each independently H, alkyl, haloalkyl, cycloalkyl, heterocycloalkyl, alkenyl, cycloalkenyl, heterocycloalkenyl, aryl, heteroaryl, aralkyl or heteroaralkyl (all optionally substituted);

or R₈ is selected as above and R₉ is:

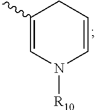

and

R₁₀ and R₁₁ are each independently H, alkyl, haloalkyl, cycloakyl, heterocycloalkyl, alkenyl, cycloalkenyl, heterocycloalkenyl, aryl, heteroaryl, aralkyl, heteroaralkyl (all optionally substituted) or OR₈.

In one embodiment, R₁-R₂ form a cycloalkyl or heterocycloalkyl.

In one embodiment, R₃-R₄ form a cycloalkyl or heterocycloalkyl.

In one embodiment, R₁₀-R₁₁ form a cycloalkyl or heterocycloalkyl.

In one embodiment, R₁-R₃ form a bond, —(CR₁₀R₁₁)ₘ— or —(CR₁₀═CR₁₁)ₘ—.

In one embodiment, R₂-R₄ form a bond, —(CR₁₀R₁₁)ₘ— or —(CR₁₀═CR₁₁)ₘ—.

In one embodiment, R₁-R₅ form a bond, —(CR₁₀R₁₁)ₘ— or —(CR₁₀═CR₁₁)ₘ—.

In one embodiment, R₂-R₆ form a bond, —(CR₁₀R₁₁)ₘ— or —(CR₁₀═CR₁₁)ₘ—.

In one embodiment, R₃-R₄ form oxo.

In one embodiment, R₃-R₅ form a bond, —(CR₁₀R₁₁)ₘ—, —(CR₁₀═CR₁₁)ₘ— or —[C(R₁₀)═C(R₁₁)—CO]—.

In one embodiment, R₄-R₆ form a bond, —(CR₁₀R₁₁)ₘ— or —(CR₁₀═CR₁₁)ₘ—.

In one embodiment, R₅-R₆ form —(CR₁₀R₁₁)ₘ— or —(CR₁₀R₁₁)ₘ—X₃—(CR₁₀R₁₁)ₘ—.

In another embodiment, the compound of Formula I is selected with the proviso that:

when X₁ is oxygen and X₂ is a bond, then n is not 0 and R₁, R₂, R₃, R₄ and R₅ are not H; and/or

when X₁ is NH and X₂ is a bond, then n is not 0 or 1 and R₁, R₂, R₃, R₄ and R₅ are not H; and/or

X₁—CR₁R₂—(CR₃R₄)ₙ—X₂—R₅ is not —O—C₁₋₃alkyl or —NH—C₁₋₃alkyl.

In another embodiment, the compound of Formula I is selected with the proviso that:

when X₁ is oxygen and X₂ is a bond, then n is not 0 or R₁, R₂, R₃, R₄ and R₅ are not H; and/or

when X₁ is NR₆ and X₂ is a bond, then n is not 0 or 1 or R₁, R₂, R₃, R₄ and R₅ are not H; and/or

—X₁—CR₁R₂—(CR₃R₄)ₙ—X₂—R₅ is not —O—C₁₋₃alkyl or —NR₆—C₁₋₃alkyl.

In another embodiment, the compound of Formula I is selected with the proviso that:

when X₁ is oxygen, X₂ is a bond, and R₁, R₂ and R₅ are H, then n is not 0; and

when X₁ is NH, X₂ is a bond, and R₁, R₂, R₃, R₄ and R₅ are H, then n is not 1.

In another embodiment, R₁ in Formula I is H or optionally substituted alkyl. In another embodiment, R₁ in Formula I is H or optionally substituted methyl. In another embodiment, R₁ in Formula I is H, CH₃ or CH₂OAc. In another embodiment, R₁ in Formula I is H or CH₃. In another embodiment, R₁ and R₅ in Formula I together form optionally substituted alkylene. In another embodiment R₁ and R₅ in Formula I together form optionally substituted ethylene or optionally substituted propylene. In another embodiment, R₁ and R₅ in Formula I together form unsubstituted ethylene. In another embodiment, R₁ and R₅ in Formula I together form unsubstituted propylene. In another embodiment, R₁ and R₅ in Formula I together form —CH(OH)CH₂—. In another embodiment, R₁ and R₅ in Formula I together form —CH(OR₁₂)CH₂—, where R₁₂ is

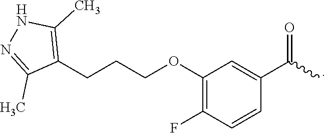

In another embodiment, R₂ in Formula I is H. In another embodiment, R₂ and R₆ in Formula I together form optionally substituted alkylene. In another embodiment, R₂ and R₆ in Formula I together form optionally substituted ethylene. In another embodiment, R₂ and R₆ in Formula I together form unsubstituted ethylene.

In another embodiment, R₃ in Formula I is H, halo or optionally substituted alkyl. In another embodiment, R₃ in Formula I is H, F or optionally substituted methyl. In another embodiment, R₃ in Formula I is H, F or unsubstituted methyl. In another embodiment, R₃ in Formula I is H or F. In another embodiment, R₃ and R₄ in Formula I together form oxo. In another embodiment, R₃ and R₅ in Formula I together form optionally substituted alkylene. In another embodiment, R₃ and R₅ in Formula I together form optionally substituted ethylene. In another embodiment, R₃ and R₅ in Formula I together form unsubstituted ethylene. In another embodiment, R₃ and R₅ in Formula I together form optionally substituted propylene. In another embodiment, R₃ and R₅ in Formula I together form unsubstituted propylene. In another embodiment, R₃ and R₅ in Formula I together form optionally substituted butylene. In another embodiment, R₃ and R₅ in Formula I together form —(CH(OH))₄—.

In another embodiment, R₄ in Formula I is H.

In another embodiment, n in Formula I is 0, 1, 2, 3 or 4. In another embodiment, n in Formula I is 1.

In another embodiment, m in Formula I is 0, 2, 3, 4 or 5. In another embodiment, m in Formula I is 2, 3, 4 or 5.

In another embodiment, X₁ in Formula I is O or NR₆. In another embodiment, X₁ in Formula I is O or NH.

In another embodiment, X₂ in Formula I is a bond, O, NH, N(alkyl), N⁺(alkyl)₂ or P⁺(aryl)₂. In another embodiment, X₂ in Formula I is a bond, O, NH, N(Me), N(Et), N⁺(Me)₂ or P⁺(Ph)₂. In another embodiment, X₂ in Formula I is a bond, O or N(Me).

In another embodiment, R₅ in Formula I is H, optionally substituted alkyl, —C(O)alkyl, heteroarylium, aryl, —COOR₉, heterocycloalkenyl or haloalkyl. In another embodiment, R₅ in Formula I is H, methyl, ethyl, —C(O)Me, pyridinium, phenyl, —COO-t-butyl, CHF₂, CF₃,

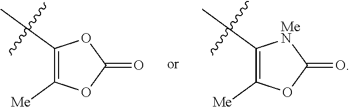 [SMILES: *c1oc(=O)oc1C ; "or" ; *c1c(C)oc(=O)n1C]

In another embodiment, R₅ in Formula I is H, alkyl, halo or heteroaryl. In another embodiment, R₅ in Formula I is H, F, CH₃, 1-imidazolyl, 2-imidazolyl or 2-pyridyl.

In another embodiment, R₅ and R₆ in Formula I together form optionally substituted alkylene. In another embodiment, R₅ and R₆ in Formula I together form unsubstituted alkylene. In another embodiment, R₅ and R₆ in Formula I together form unsubstituted propylene, butylene or pentylene.

In another embodiment, R₆ in Formula I is H or alkyl. In another embodiment, R₆ in Formula I is H, methyl or ethyl. In another embodiment, R₆ in Formula I is H or methyl.

In another embodiment, R₇ in Formula I is H or alkyl. In another embodiment, R₇ in Formula I is methyl.

In another embodiment, R₈ in Formula I is H or alkyl. In another embodiment, R₈ in Formula I is methyl.

In another embodiment, R₉ in Formula I is H or alkyl. In another embodiment, R₉ in Formula I is methyl.

In another embodiment, R₁₀ in Formula I is H, OH or alkyl. In another embodiment, R₁₀ in Formula I is H or OH. In another embodiment, R₁₀ in Formula I is H.

In another embodiment, R₁₁ in Formula I is H or alkyl. In another embodiment, R₁₁ in Formula I is H.

In another embodiment, provided herein is a compound for use in the compositions and methods provided herein having the structure of Formula II, or pharmaceutically acceptable salt or solvate thereof:

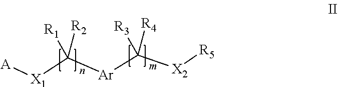

wherein:

A is

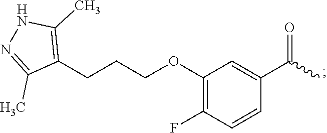

X₁ is O, S or NR₆;

X₂ is a bond, O, S, NR₆, N⁺R₆R₇ or P⁺(Ar)₂;

X₃ is O, S or NR₆;

n is an integer from 0-6;

m is an integer from 0-6;

each Ar is independently cycloalkylene, heterocycloalkylene, arylene, heteroarylene or heteroarylenium (all optionally substituted);

R₁, R₂, R₃, R₄, R₅, R₆ and R₇ are each independently H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OH, OR₈, COR₉, COOR₉, —(CR₁₀R₁₁)ₘX₃R₈, or —(CR₁₀R₁₁)ₘX₃COR₉;

R₈ and R₉ are each independently H, alkyl, haloalkyl, cycloalkyl, heterocycloalkyl, alkenyl, cycloalkenyl, heterocycloalkenyl, aryl, heteroaryl, aralkyl or heteroaralkyl (all optionally substituted);

or R₈ is selected as above and R₉ is

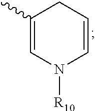

and

R₁₀ and R₁₁ are independently H, alkyl, haloalkyl, cycloakyl, heterocycloalkyl, alkenyl, cycloalkenyl, heterocycloalkenyl, aryl, heteroaryl, aralkyl, heteroaralkyl (all optionally substituted), OH or OR₈.

In one embodiment, R₁-R₂ form a cycloalkyl or heterocycloalkyl.

In one embodiment, R₃-R₄ form a cycloalkyl or heterocycloalkyl.

In one embodiment, R₁₀-R₁₁ form a cycloalkyl or heterocycloalkyl.

In one embodiment, R₁-R₃ form a bond, —(CR₁₀R₁₁)ₘ— or —(CR_{10═}CR₁₁)ₘ—.

In one embodiment, R₂-R₄ form a bond, —(CR₁₀R₁₁)ₘ— or —(CR_{10═}CR₁₁)ₘ—.

In one embodiment, R₁-R₅ form a bond, —(CR₁₀R₁₁)ₘ— or —(CR_{10═}CR₁₁)ₘ—.

In one embodiment, R₂-R₆ form a bond, —(CR₁₀R₁₁)ₘ— or —(CR_{10═}CR₁₁)ₘ—.

In one embodiment, R₃-R₅ form a bond, —(CR₁₀R₁₁)ₘ—, —(CR_{10═}CR₁₁)ₘ or —[C(R₁₀)═C(R₁₁)—CO]—.

In one embodiment, R₄-R₆ form a bond, —(CR₁₀R₁₁)ₘ— or —(CR_{10═}CR₁₁)ₘ—.

In one embodiment, R₅-R₆ form —(CR₁₀R₁₁)ₘ— or —(CR₁₀R₁₁)ₘ—X₃—(CR₁₀R₁₁)ₘ—.

In another embodiment, the compounds for use in the compositions and methods provided herein have Formula II, or pharmaceutically acceptable salt or solvate thereof, wherein:

A is

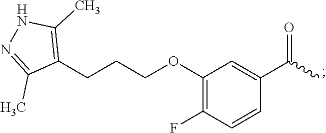

X₁ is O, S or NR₆;

X₂ is a bond, O, S, NR₆, N⁺R₆R₇ or P⁺(Ar)₂;

X₃ is O, S or NR₆;

n is an integer from 0-6;

m is an integer from 0-6;

each Ar is independently arylene, heteroarylene or heteroarylenium (all optionally substituted);

R₁, R₂, R₃, R₄, R₅, R₆ and R₇ are each independently H, alkyl, haloalkyl, cycloalkyl, heterocycloalkyl, alkenyl, cycloalkenyl, heterocycloalkenyl, aryl, heteroaryl, aralkyl, heteroaralkyl (all optionally substituted), OH, OR₈, COR₉, —(CR₁₀R₁₁)ₘX₃R₈, or —(CR₁₀R₁₁)ₘX₃COR₉;

R₈ and R₉ are each independently H, alkyl, haloalkyl, cycloalkyl, heterocycloalkyl, alkenyl, cycloalkenyl, heterocycloalkenyl, aryl, heteroaryl, aralkyl or heteroaralkyl (all optionally substituted);

or R₈ is selected as above and R₉ is

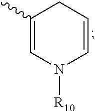

and

R₁₀ and R₁₁ are independently H, alkyl, haloalkyl, cycloakyl, heterocycloalkyl, alkenyl, cycloalkenyl, heterocycloalkenyl, aryl, heteroaryl, aralkyl, heteroaralkyl (all optionally substituted), OH or OR₈.

In one embodiment, R₁-R₂ form a cycloalkyl or heterocycloalkyl.

In one embodiment, R₃-R₄ form a cycloalkyl or heterocycloalkyl.

In one embodiment, R₁₀-R₁₁ form a cycloalkyl or heterocycloalkyl.

In one embodiment, R₁-R₃ form a bond, —(CR₁₀R₁₁)ₘ— or —(CR₁₀═CR₁₁)ₘ—.

In one embodiment, R₂-R₄ form a bond, —(CR₁₀R₁₁)ₘ— or —(CR₁₀═CR₁₁)ₘ—.

In one embodiment, R₁-R₅ form a bond, —(CR₁₀R₁₁)ₘ— or —(CR₁₀═CR₁₁)ₘ—.

In one embodiment, R₂-R₆ form a bond, —(CR₁₀R₁₁)ₘ— or —(CR₁₀═CR₁₁)ₘ—.

In one embodiment, R₃-R₅ form a bond, —(CR₁₀R₁₁)ₘ, —(CR₁₀═CR₁₁)ₘ— or —[C(R₁₀)═C(R₁₁)—CO]—.

In one embodiment, R₄-R₆ form a bond, —(CR₁₀R₁₁)ₘ— or —(CR₁₀═CR₁₁)ₘ—.

In one embodiment, R₅-R₆ form —(CR₁₀R₁₁)ₘ— or —(CR₁₀R₁₁)ₘ—X₃—(CR₁₀R₁₁)ₘ—.

In another embodiment, X₁ in Formula II is O.

In another embodiment, n in Formula II is 0.

In another embodiment, Ar in Formula II is arylene or heterocyloalkylene. In another embodiment, Ar in Formula II is phenylene or bridged bycyclic heterocyloalkylene. In another embodiment, Ar in Formula II is 1,4- or 1,3-phenylene, or 1- or 2-azabicyclo[2.2.2]octanylene.

In another embodiment, R₃ in Formula II is H or COOR₉. In another embodiment, R₃ in Formula II is H or COO-aralkyl. In another embodiment, R₃ in Formula II is H or COOBn.

In another embodiment, R₄ in Formula II is H.

In another embodiment, n in Formula II is 0.

In another embodiment, m in Formula II is 0 or 1.

In another embodiment, X₂ in Formula II is a bond or NR₆. In another embodiment, X₂ in Formula II is a bond or NH.

In another embodiment, R₅ in Formula II is H, alkyl or COOR₉. In another embodiment, R₅ in Formula II is H, methyl or COO-alkyl. In another embodiment, R₅ in Formula II is H, methyl or COO-t-butyl.

In some embodiments, the compound provided herein for use in the compositions and methods provided herein is selected from the compounds in Table 1.

TABLE 1

| Compound # | Structure |
|---|---|
| 1 | 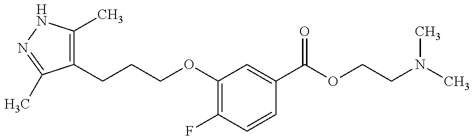 [SMILES: Cc1n[nH]c(C)c1CCCOc1cc(C(=O)OCCN(C)C)ccc1F] |
| 2 | 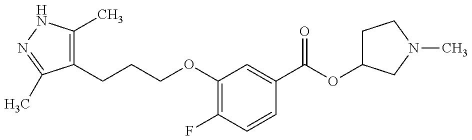 [SMILES: Cc1n[nH]c(C)c1CCCOc1cc(C(=O)OC2CCN(C)C2)ccc1F] |
| 3 | 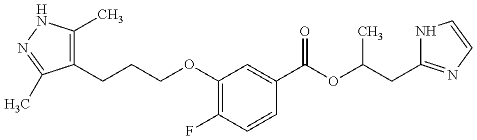 [SMILES: Cc1n[nH]c(C)c1CCCOc1cc(C(=O)OC(C)Cc2ncc[nH]2)ccc1F] |
| 4 | 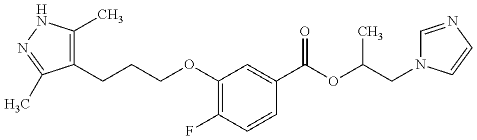 [SMILES: Cc1n[nH]c(C)c1CCCOc1cc(C(=O)OC(C)Cn2ccnc2)ccc1F] |
| 5 | 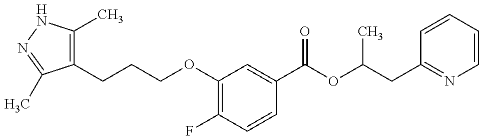 [SMILES: Cc1n[nH]c(C)c1CCCOc1cc(C(=O)OC(C)Cc2ccccn2)ccc1F] |
| 6 | 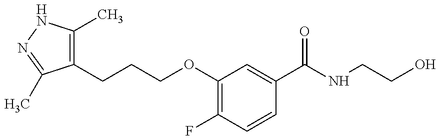 [SMILES: Cc1n[nH]c(C)c1CCCOc1cc(C(=O)NCCO)ccc1F] |
| 7 | 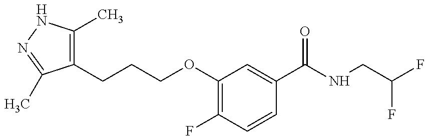 [SMILES: Cc1n[nH]c(C)c1CCCOc1cc(C(=O)NCC(F)F)ccc1F] |
| 8 | 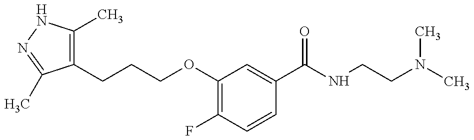 [SMILES: Cc1n[nH]c(C)c1CCCOc1cc(C(=O)NCCN(C)C)ccc1F] |

### III. Exemplary Syntheses

The compounds provided herein may be prepared by methods well known to those of skill in the art. For example, the compounds may be prepared under standard coupling conditions, e.g., DCC/DMAP, by reacting acoramidis with an amine or alcohol. General methods of preparation are as follows:

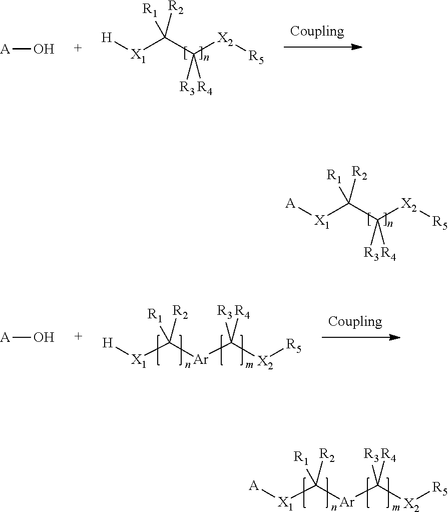

### IV. Methods of Use

The compounds provided herein are useful in treating transthyretin amyloid disease. Without being bound by any theory, the compounds act by inhibiting and preventing TTR aggregation and/or amyloid formation by stabilizing native tetrameric TTR structure therefore preventing dissociation of the tetramer TTR and the deposition of TTR amyloid fibrils in all relevant tissues for TTR amyloid diseases. The transthyretin amyloid disease can be, for example, familial amyloid polyneuropathy (ATTR-FAP), familial amyloid cardiomyopathy (ATTR-FAC), senile systemic amyloidosis and TTR oculoleptomeningeal amyloidosis (ATTR-OLMA).

Prodrugs of TTR stabilizers with good brain and eye penetration should fulfill the current unmet medical need (ocular and cerebral amyloid angiopathies) as an oral drug, by parenteral, intravenous or other injectable delivery, or by local delivery (such as topical eye or intranasal delivery). Tafamidis and diflunisal, two TTR stabilizers with demonstrated clinical efficacy to treat peripheral TTR amyloidosis, are very poor brain and eye penetrating drugs Similarly, acoramidis (AG10), a TTR stabilizer currently in clinical evaluation, has poor brain penetration. Compounds provided herein have improved brain penetration by systemic administration and deliver increased levels of TTR stabilizer in the brain. Because the Blood brain barrier (BBB), the blood CSF barrier (BCSFB) and the blood-ocular barrier (BOB) share similarities in microscopic structure, it is recognized in the art that one site may serve as a pharmacokinetic surrogate for the others. Therefore, one of skill in the art would expect a brain penetrating compound to penetrate the eye as well.

Compounds described herein can also be delivered locally to the eye or by intranasal delivery.

Compounds described herein may be useful for treating human patients with TTR oculoleptomeningeal amyloidosis in ATTR patients, including but not restricted to ATTR-OLMA and ATTR-FAP patients.

Combination therapy may include, but is not limited to liver transplantation, TTR stabilizer such as tafamidis, knock-down therapies such as anti-TTR siRNA and antisense (patisiran and inotersen).

In another embodiment, provided herein are processes and novel intermediates which are useful for preparing compounds provided herein. In other embodiments, methods for synthesis, analysis, separation, isolation, purification, characterization, and testing of the compounds provided herein are provided.

### V. Methods of Treating Disease

In another embodiment, a method of treating a subject with peripheral TTR amyloidosis is provided. The method includes administering to a subject having peripheral TTR amyloidosis an effective amount of a compound of Formula I or II. Diseases contemplated in the practice of the methods disclosed herein include familial amyloid polyneuropathy (ATTR-FAP), familial amyloid cardiomyopathy (ATTR-FAC), senile systemic amyloidosis and diseases related to TTR oculoleptomeningeal amyloidosis in ATTR patients, including but not restricted to ATTR-OLMA and ATTR-FAP patients.

### VI. Pharmaceutical Compositions

In another embodiment, provided herein are pharmaceutical compositions. The pharmaceutical composition includes a pharmaceutically acceptable excipient and a compound provided herein (e.g., Formula I or II).

The pharmaceutical compositions provided herein are typically used to treat a disorder or condition using TTR stabilizer therapies.

In an exemplary embodiment, the pharmaceutical composition includes from 1 μg to 2000 mg of a compound disclosed herein, e.g., 1 μg to 1 mg, 1 mg to 10 mg, 1 mg to 100 mg, 1 mg to 1000 mg, 1 mg to 1500 mg, or 1 mg to 2000 mg.

### A. Formulations

The compounds provided herein can be formulated and administered in a wide variety of oral, parenteral and topical dosage forms. Oral preparations include tablets, pills, powder, dragees, capsules, liquids, lozenges, gels, syrups, slurries, suspensions, etc., suitable for ingestion by the patient. The compounds provided herein can also be administered by injection, that is, intravenously, intramuscularly, intracutaneously, subcutaneously, intraduodenally, or intraperitoneally. Also, the compounds provided herein can be administered by inhalation, for example, intranasally. Additionally, the compounds provided herein can be administered transdermally. The compounds provided herein can also be administered by in intraocular, intravaginal, and intrarectal routes including suppositories, insufflation, powders and aerosol formulations (for examples of steroid inhalants, see Rohatagi, J. Clin. Pharmacol. 35:1187-1193, 1995; Tjwa, Ann. Allergy Asthma Immunol. 75:107-111, 1995). Thus, the pharmaceutical compositions provided herein may be adapted for oral administration. In some embodiments, the pharmaceutical composition is in the form of a tablet. Moreover, provided herein are pharmaceutical compositions including a pharmaceutically acceptable carrier or excipient and either a compound provided herein, or a pharmaceutically acceptable salt of a compound provided herein.

For preparing pharmaceutical compositions from the compounds provided herein, pharmaceutically acceptable carriers can be either solid or liquid. Solid form preparations include powders, tablets, pills, capsules, cachets, suppositories, and dispersible granules. A solid carrier can be one or more substances, which may also act as diluents, flavoring agents, binders, preservatives, tablet disintegrating agents, or an encapsulating material. Details on techniques for formulation and administration are well described in the scientific and patent literature, see, e.g., the latest edition of REMINGTON'S PHARMACEUTICAL SCIENCES, Maack Publishing Co, Easton PA (“Remington's”).

In powders, the carrier is a finely divided solid, which is in a mixture with the finely divided compound provided herein. In tablets, the compound provided herein is mixed with the carrier having the necessary binding properties in suitable proportions and compacted in the shape and size desired.

The powders and tablets preferably contain from 5% or 10% to 70% of the compound provided herein. Suitable carriers are magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. The term “preparation” is intended to include the formulation of the compound provided herein with encapsulating material as a carrier providing a capsule in which the compound provided herein with or without other carriers, is surrounded by a carrier, which is thus in association with it. Similarly, cachets and lozenges are included. Tablets, powders, capsules, pills, cachets, and lozenges can be used as solid dosage forms suitable for oral administration.

Suitable solid excipients are carbohydrate or protein fillers include, but are not limited to sugars, including lactose, sucrose, mannitol, or sorbitol; starch from corn, wheat, rice, potato, or other plants; cellulose such as methyl cellulose, hydroxypropylmethyl-cellulose, or sodium carboxymethylcellulose; and gums including arabic and tragacanth; as well as proteins such as gelatin and collagen. If desired, disintegrating or solubilizing agents may be added, such as the cross-linked polyvinyl pyrrolidone, agar, alginic acid, or a salt thereof, such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of compound provided herein (i.e., dosage). Pharmaceutical preparations provided herein can also be used orally using, for example, push-fit capsules made of gelatin, as well as soft, sealed capsules made of gelatin and a coating such as glycerol or sorbitol. Push-fit capsules can contain compounds of Formulae I or II mixed with a filler or binders such as lactose or starches, lubricants such as talc or magnesium stearate, and, optionally, stabilizers. In soft capsules, the compounds may be dissolved or suspended in suitable liquids, such as fatty oils, liquid paraffin, or liquid polyethylene glycol with or without stabilizers.

For preparing suppositories, a low melting wax, such as a mixture of fatty acid glycerides or cocoa butter, is first melted and the active component is dispersed homogeneously therein, as by stirring. The molten homogeneous mixture is then poured into convenient sized molds, allowed to cool, and thereby to solidify.

Liquid form preparations include solutions, suspensions, and emulsions, for example, water or water/propylene glycol solutions. For parenteral injection, liquid preparations can be formulated in solution in aqueous polyethylene glycol solution.

Aqueous solutions suitable for oral use can be prepared by dissolving the compound provided herein in water and adding suitable colorants, flavors, stabilizers, and thickening agents as desired. Aqueous suspensions suitable for oral use can be made by dispersing the finely divided compound provided herein in water with viscous material, such as natural or synthetic gums, resins, methylcellulose, sodium carboxymethylcellulose, hydroxypropylmethylcellulose, sodium alginate, polyvinylpyrrolidone, gum tragacanth and gum acacia, and dispersing or wetting agents such as a naturally occurring phosphatide (e.g., lecithin), a condensation product of an alkylene oxide with a fatty acid (e.g., polyoxyethylene stearate), a condensation product of ethylene oxide with a long chain aliphatic alcohol (e.g., heptadecaethylene oxycetanol), a condensation product of ethylene oxide with a partial ester derived from a fatty acid and a hexitol (e.g., polyoxyethylene sorbitol mono-oleate), or a condensation product of ethylene oxide with a partial ester derived from fatty acid and a hexitol anhydride (e.g., polyoxyethylene sorbitan mono-oleate). The aqueous suspension can also contain one or more preservatives such as ethyl or n-propyl p-hydroxybenzoate, one or more coloring agents, one or more flavoring agents and one or more sweetening agents, such as sucrose, aspartame or saccharin. Formulations can be adjusted for osmolarity.

Also included are solid form preparations, which are intended to be converted, shortly before use, to liquid form preparations for oral administration. Such liquid forms include solutions, suspensions, and emulsions. These preparations may contain, in addition to the compound provided herein, colorants, flavors, stabilizers, buffers, artificial and natural sweeteners, dispersants, thickeners, solubilizing agents, and the like.

Oil suspensions can be formulated by suspending a compound provided herein in a vegetable oil, such as arachis oil, olive oil, sesame oil or coconut oil, or in a mineral oil such as liquid paraffin; or a mixture of these. The oil suspensions can contain a thickening agent, such as beeswax, hard paraffin or cetyl alcohol. Sweetening agents can be added to provide a palatable oral preparation, such as glycerol, sorbitol or sucrose. These formulations can be preserved by the addition of an antioxidant such as ascorbic acid. As an example of an injectable oil vehicle, see Minto, J. Pharmacol. Exp. Ther. 281:93-102, 1997. The pharmaceutical formulations provided herein can also be in the form of oil-in-water emulsions. The oily phase can be a vegetable oil or a mineral oil, described above, or a mixture of these. Suitable emulsifying agents include naturally-occurring gums, such as gum acacia and gum tragacanth, naturally occurring phosphatides, such as soybean lecithin, esters or partial esters derived from fatty acids and hexitol anhydrides, such as sorbitan mono-oleate, and condensation products of these partial esters with ethylene oxide, such as polyoxyethylene sorbitan mono-oleate. The emulsion can also contain sweetening agents and flavoring agents, as in the formulation of syrups and elixirs. Such formulations can also contain a demulcent, a preservative, or a coloring agent.

The compounds provided herein can be delivered by transdermally, by a topical route, formulated as applicator sticks, solutions, suspensions, emulsions, gels, creams, ointments, pastes, jellies, paints, powders, and aerosols.

The compounds provided herein can also be delivered as microspheres for slow release in the body. For example, microspheres can be administered via intradermal injection of drug-containing microspheres, which slowly release subcutaneously (see Rao, J. Biomater Sci. Polym. Ed. 7:623-645, 1995); as biodegradable and injectable gel formulations (see, e.g., Gao Pharm. Res. 12:857-863, 1995); or, as microspheres for oral administration (see, e.g., Eyles, J. Pharm. Pharmacol. 49:669-674, 1997). Both transdermal and intradermal routes afford constant delivery for weeks or months.

The compounds provided herein can be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preparation may be a lyophilized powder in 1 mM-50 mM histidine, 0.1%-2% sucrose, 2%-7% mannitol at a pH range of 4.5 to 5.5, that is combined with buffer prior to use.

In another embodiment, the compounds provided herein are useful for parenteral administration, such as intravenous (IV) administration or administration into a body cavity or lumen of an organ. The formulations for administration will commonly comprise a solution of the compound provided herein dissolved in a pharmaceutically acceptable carrier. Among the acceptable vehicles and solvents that can be employed are water and Ringer's solution, an isotonic sodium chloride. In addition, sterile fixed oils can conventionally be employed as a solvent or suspending medium. For this purpose, any bland fixed oil can be employed including synthetic mono- or diglycerides. In addition, fatty acids such as oleic acid can likewise be used in the preparation of injectables. These solutions are sterile and generally free of undesirable matter. These formulations may be sterilized by conventional, well known sterilization techniques. The formulations may contain pharmaceutically acceptable auxiliary substances as required to approximate physiological conditions such as pH adjusting and buffering agents, toxicity adjusting agents, e.g., sodium acetate, sodium chloride, potassium chloride, calcium chloride, sodium lactate and the like. The concentration of the compound provided herein in these formulations can vary widely, and will be selected primarily based on fluid volumes, viscosities, body weight, and the like, in accordance with the particular mode of administration selected and the patient's needs. For IV administration, the formulation can be a sterile injectable preparation, such as a sterile injectable aqueous or oleaginous suspension. This suspension can be formulated according to the known art using those suitable dispersing or wetting agents and suspending agents. The sterile injectable preparation can also be a sterile injectable solution or suspension in a nontoxic parenterally-acceptable diluent or solvent, such as a solution of 1,3-butanediol.

In another embodiment, the compound provided herein can be delivered by the use of liposomes which fuse with the cellular membrane or are endocytosed, i.e., by employing ligands attached to the liposome, or attached directly to the compound provided herein, that bind to surface membrane protein receptors of the cell resulting in endocytosis. By using liposomes, particularly where the liposome surface carries ligands specific for target cells, or are otherwise preferentially directed to a specific organ, one can focus the delivery of the compound into the target cells in vivo. (See, e.g., Al-Muhammed, J. Microencapsul. 13:293-306, 1996; Chonn, Curr. Opin. Biotechnol. 6:698-708, 1995; Ostro, Am. J. Hosp. Pharm. 46:1576-1587, 1989).

The pharmaceutical preparation is preferably in unit dosage form. In such form the preparation is subdivided into unit doses containing appropriate quantities of the compound provided herein. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

The quantity of compound provided herein in a unit dose preparation may be varied or adjusted from 0.1 mg to 10000 mg, more typically 1.0 mg to 1000 mg, most typically 10 mg to 500 mg, according to the particular application and the potency of the compound provided herein. The composition can, if desired, also contain other compatible therapeutic agents.

Compounds provided herein may be metabolized by cells and then converted to the active TTR stabilizer.

### B. Effective Dosages

Pharmaceutical compositions provided herein include compositions wherein the compound provided herein is contained in a therapeutically effective amount, i.e., in an amount effective to achieve its intended purpose. The actual amount effective for a particular application will depend on the condition being treated. For example, when administered in methods to treat TTR related conditions, such compositions will contain an amount of compound provided herein effective to achieve the desired result.

The dosage and frequency (single or multiple doses) of compound provided herein administered can vary depending upon a variety of factors, including route of administration; size, age, sex, health, body weight, body mass index, and diet of the recipient; nature and extent of symptoms of the disease being treated; presence of other diseases or other health-related problems; kind of concurrent treatment; and complications from any disease or treatment regimen. Other therapeutic regimens or agents can be used in conjunction with the methods and compounds provided herein.

For any compound provided herein, the therapeutically effective amount can be initially determined from cell culture assays. Target concentrations will be those concentrations of compound provided herein that are capable of decreasing viral activity as measured, for example, using the methods provided herein.

Therapeutically effective amounts for use in humans may be determined from animal models. For example, a dose for humans can be formulated to achieve a concentration that has been found to be effective in animals. The dosage in humans can be adjusted by monitoring viral inhibition and adjusting the dosage upwards or downwards, as described above.

Dosages may be varied depending upon the requirements of the patient and the compound being employed. The dose administered to a patient, in the context of the present disclosure, should be sufficient to affect a beneficial therapeutic response in the patient over time. The size of the dose also will be determined by the existence, nature, and extent of any adverse side effects. Generally, treatment is initiated with smaller dosages, which are less than the optimum dose of the compound provided herein. Thereafter, the dosage is increased by small increments until the optimum effect under circumstances is reached. In one embodiment, the dosage range is 0.001% to 10% w/v. In another embodiment, the dosage range is 0.1% to 5% w/v.

Dosage amounts and intervals can be adjusted individually to provide levels of the administered compound provided herein effective for the particular clinical indication being treated. This will provide a therapeutic regimen that is commensurate with the severity of the individual's disease state.

Utilizing the teachings provided herein, an effective prophylactic or therapeutic treatment regimen can be planned that does not cause substantial toxicity and yet is entirely effective to treat the clinical symptoms demonstrated by the particular patient. This planning should involve the careful choice of compound provided herein by considering factors such as compound potency, relative bioavailability, patient body weight, presence and severity of adverse side effects, mode of administration, and the toxicity profile of the selected agent.

### VII. Examples

The examples below are meant to illustrate certain embodiments provided herein, and not to limit the scope of this disclosure.

Abbreviations: DCM—Dichloromethane; h—hour; TEA—Triethylamine; TLC—thin layer chromatography

The following references provide synthetic and analytical procedures that would be useful to those of skill in the art in preparing and analyzing the compounds provided here. Each reference disclosed herein is incorporated by reference in its entirety for all purposes.
1. Miller, et al. Enthalpy-Driven Stabilization of Transthyretin by AG10 Mimics a Naturally Occurring Genetic Variant That Protects from Transthyretin Amyloidosis J. Med. Chem. 2018, 61, 7862-7876
2. WO 2018/151815 A1
3. WO 2014/100227 A1

¹H NMR Conditions: Instrument Type: AVANCE III 400 or AVANCE III 400 HD or AVANCE NEO; Probe Type: 5 mm PABBO BB or 5 mm CPP BBO; Frequency (MHz): 400.1300; Temperature (° C.): 27.

### LCMS Methods:

Method 1: Instrument: SHIMADZU LCMS-2020; Column: Kinetex EVO C18 2.1×30 mm, 5 um; Mobile Phase: A: 0.0375% TFA in water (v/v), B: 0.01875% TFA in Acetonitrile (v/v); Gradient: 0.0 min 5% B→0.8 min 95% B→1.2 min 95% B→1.21 min 5% B→1.55 min 5% B; Flow: 1.5 mL/min; Column Temp: 50° C.; Detector: PDA (220 & 254 nm). Ionization source: ESI.

Method 2: Instrument: SHIMADZU LCMS-2020; Column: Kinetex EVO C18 2.1×30 mm, 5 μm; Mobile Phase: A: 0.025% NH₃·H₂O in water (v/v), B: Acetonitrile; Gradient: 0.0 min 5% B→0.8 min 95% B→1.2 min 95% B→1.21 min 5% B→1.55 min 5% B; Flow: 1.5 mL/min; Column Temp: 50° C.; Detector: PDA (220 & 254 nm). Ionization source: ESI.

### HPLC Methods

Method 1: Instrument: SHIMADZU LC-20AB; Column: Kinetex C18 LC Column 4.6×50 mm, 5 μm; Mobile Phase: A: 0.0375% TFA in water (v/v), B: 0.01875% TFA in Acetonitrile (v/v); Gradient: 0.0 min 10% B→2.40 min 80% B→3.70 min 80% B→3.71 min 10% B→4.00 min 10% B; Flow: 1.5 mL/min; Column Temp: 50° C.; Detector: PDA (220 nm & 215 nm & 254 nm).

Method 2: Instrument: SHIMADZU LC-20AB; Column: XBridge C18, 2.1×50 mm, 5 μm; Mobile Phase: A: 0.025% NH₃·H₂O in water (v/v), B: Acetonitrile; Gradient: 0.0 min 10% B→4.20 min 80% B→5.30 min 80% B→5.31 min 10% B→6.00 min 10% B; Flow: 0.8 mL/min; Column Temp: 40° C.; Detector: PDA (220 nm & 215 nm & 254 nm).

Method 3: Instrument: SHIMADZU LC-20AB; Column: XBridge C18, 2.1×50 mm, 3.5 μm; Mobile Phase: A: 0.025% NH₃·H₂O in water (v/v), B: Acetonitrile; Gradient: 0.0 min 30% B→3.00 min 90% B→3.50 min 90% B→3.51 min 30% B→4.00 min 30% B; Flow: 1.2 mL/min; Column Temp: 50° C.; Detector: PDA (220 nm & 215 nm & 254 nm).

### Example 1

### Compound 1: 2-(dimethylamino)ethyl 3-(3-(3,5-dimethyl-1H-pyrazol-4-yl)propoxy)-4-fluorobenzoate

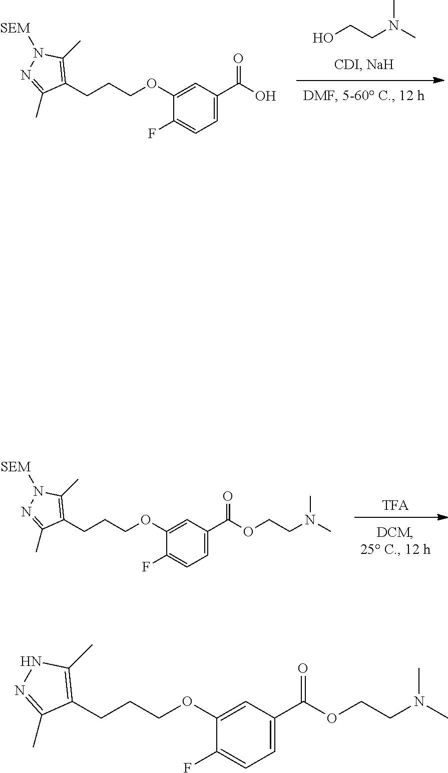

Step 1: 2-(dimethylamino)ethyl 3-(3-(3,5-dimethyl-1-((2-(trimethylsilypethoxy)methyl)-1H-pyrazol-4-yl)propoxy)-4-fluorobenzoate

To a solution of 3-(3-(3,5-dimethyl-1-((2-(trimethylsilyl)ethoxy)methyl)-1H-pyrazol-4-yl)propoxy)-4-fluorobenzoic acid (430 mg, 1.02 mmol) in DMF (10 mL) was added CDI (247.50 mg, 1.53 mmol), the mixture was stirred at 25° C. for 0.5 h. Then the mixture cooled to 5° C. was added 2-(dimethylamino)ethanol (136.06 mg, 1.53 mmol) and NaH (81.41 mg, 2.04 mmol, 60% purity). The mixture was stirred at 60° C. for 12 h. The mixture was quenched with NH₄Cl solution (20 mL). The aqueous phase was extracted with EtOAc (20 mL×2). The combined organic phase was washed with brine (10 mL), dried with anhydrous Na₂SO₄, filtered and concentrated in vacuo to afford the title compound (330 mg, 521.38 μmol, 51.2% yield, 78% purity) as a colorless oil.

LCMS: m/z 494.2 [M+H]⁺.

Step 2: 2-(dimethylamino)ethyl 3-(3-(3,5-dimethyl-1H-pyrazol-4-yl)propoxy)-4-fluorobenzoate

To a solution of 2-(dimethylamino)ethyl 3-(3-(3,5-dimethyl-1-((2-(trimethylsilyl)ethoxy)methyl)-1H-pyrazol-4-yl)propoxy)-4-fluorobenzoate (330 mg, 521.38 mol) in DCM (5 mL) was added TFA (2.31 g, 20.26 mmol, 1.5 mL). The mixture was stirred at 25° C. for 12 h. The mixture was poured into NH₄Cl solution (50 mL). The aqueous phase was extracted with DCM (20 mL×2). The combined organic phase was washed with brine (10 mL), dried with Na₂SO₄, filtered and concentrated in vacuum. The residue was purified by prep-HPLC (column: Phenomenex Gemini-NX C18 75×30 mm×3 um; mobile phase: (water (10 mM NH₄HCO₃)-ACN); B %: 22%-52%, 8 min) and prep-HPLC (column: 3 Phenomenex Luna C18 75×30 mm×3 μm; mobile phase: (water (0.05% HCl)-ACN); B %: 9%-29%, 6.5 min) to afford the title compound (56.77 mg, 154.45 μmol, 29.6% yield, 98.9% purity) as an off-white solid.

LCMS: m/z 364.1 [M+H]⁺.

¹H NMR (400 MHz, CDCl₃) δ=12.89-12.07 (m, 1H), 7.68 (br d, J=7.2 Hz, 2H), 7.14 (br t, J=9.6 Hz, 1H), 4.

### Example 2

### Compound 2: 1-methylpyrrolidin-3-yl 3-(3-(3,5-dimethyl-1H-pyrazol-4-yl)propoxy)-4-fluorobenzoate

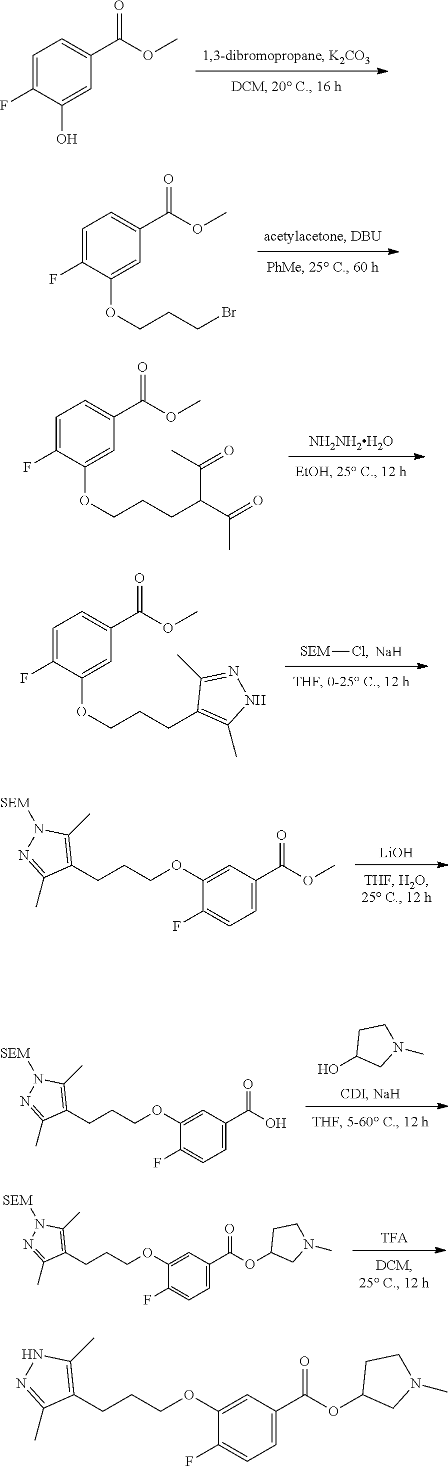

Step 1: methyl 3-(3-bromopropoxy)-4-fluorobenzoate

To a solution of methyl 4-fluoro-3-hydroxy-benzoate (2 g, 11.76 mmol) in DMF (50 mL) was added 1,3-dibromopropane (18.99 g, 94.04 mmol, 9.59 mL) and K2CO3 (2.44 g, 17.63 mmol). The mixture was stirred at 20° C. for 16 h. The mixture was added to water (200 mL). The aqueous phase was extracted with ethyl acetate (100 mL×3). The combined organic phase was washed with brine (50 mL×3), dried with anhydrous Na2SO4, filtered and concentrated in vacuum. The residue was purified by column chromatography (SiO2, Petroleum ether/Ethyl acetate=1/0 to 30/1) to afford the title compound (3.4 g, 11.68 mmol, 99.4% yield) as a colorless oil.

LCMS: m/z 293.0 [M+1-1]+.

Step 2: methyl 3-((4-acetyl-5-oxohexyl)oxy)-4-fluorobenzoate

To a solution of methyl 3-(3-bromopropoxy)-4-fluorobenzoate (3.4 g, 11.68 mmol) in toluene (30 mL) was added pentane-2,4-dione (2.34 g, 23.36 mmol, 2.40 mL) and DBU (3.56 g, 23.36 mmol, 3.52 mL). The mixture was stirred at 25° C. for 60 h. The mixture was concentrated in vacuum to afford a residue. The residue was purified by column chromatography (SiO2, Petroleum ether/Ethyl acetate=1/0 to 5/1) to afford the title compound (1.6 g, 5.16 mmol, 44.2% yield) as a yellow oil.

¹H NMR (400 MHz, CD₃OD) δ=7.77-7.62 (m, 2H), 7.22-7.19 (m, 1H), 4.27 (t, J=6.0 Hz, 1H), 4.18-4.05 (m, 2H), 3.91 (s, 3H), 2.31-2.26 (m, 2H), 2.26-2.22 (m, 2H), 2.17 (d, J=8.2 Hz, 3H).

Step 3: methyl 3-(3-(3,5-dimethyl-1H-pyrazol-4-yl)propoxy)-4-fluorobenzoate

To a solution of methyl methyl 3-((4-acetyl-5-oxohexyl)oxy)-4-fluorobenzoate (1.5 g, 4.83 mmol) in EtOH (20 mL) was added NH₂NH₂·H₂O (241.98 mg, 4.83 mmol, 234.93 μL). The reaction mixture was heated to 25° C. and stirred for 12 h. The mixture was concentrated in vacuum to afford the title compound (1.5 g, crude) as a yellow oil.

LCMS: m/z 307.1 [M+H]+.

¹H NMR (400 MHz, CDCl₃) δ=7.72-7.57 (m, 2H), 7.12 (ddd, J=3.4, 8.4, 10.8 Hz, 1H), 4.25 (t, J=6.0 Hz, 1H), 4.02 (t, J=6.0 Hz, 1H), 3.90 (d, J=2.6 Hz, 3H), 2.63 (t, J=7.2 Hz, 1H), 2.33 (s, 3H), 2.28 (s, 3H), 2.14-2.06 (m, 1H), 1.99 (br t, J=6.8 Hz, 1H), 1.24 (t, J=7.0 Hz, 1H).

Step 4: methyl 3-(3-(3,5-dimethyl-1-((2-(trimethylsilypethoxy)methyl)-1H-pyrazol-4-yl)propoxy)-4-fluorobenzoate

To a solution of methyl methyl 3-(3-(3,5-dimethyl-1H-pyrazol-4-yl)propoxy)-4-fluorobenzoate (320 mg, 1.04 mmol) in THF (5 mL) was added SEMCl (261.24 mg, 1.57 mmol) and NaH (62.67 mg, 1.57 mmol, 60% purity) at 0° C. The mixture was stirred at 25° C. for 12 h. The mixture was quenched by NH₄Cl solution (20 mL), extracted with ethyl acetate (20 mL×2). The combined organic phase was washed brine (10 mL), dried with anhydrous Na₂SO₄, filtered and concentrated in vacuum. The residue was purified by column chromatography (SiO₂, Petroleum ether/Ethyl acetate=1/0 to 5/1) to afford the title compound (300 mg, 687.14 μmol, 65.8% yield) as a colorless oil.

LCMS: m/z 437.2 [M+H]+.

Step 5: 3-(3-(3,5-dimethyl-1-((2-(trimethylsilyl)ethoxy)methyl)-1H-pyrazol-4-y1)propoxy)-4-fluorobenzoic acid

To a solution of methyl 3-(3-(3,5-dimethyl-1-((2-(trimethylsilyl)ethoxy)methyl)-1H-pyrazol-4-yl)propoxy)-4-fluorobenzoate (300 mg, 687.14 μmol) in THF (4 mL) was added a solution of LiOH·H₂O (144.16 mg, 3.44 mmol) in H₂O (2 mL). The mixture was stirred at 25° C. for 12 h. The mixture was poured into water (20 mL). The aqueous phase was extracted with ethyl acetate (20 mL). The aqueous phase was adjusted to pH=2 with HCl (1 N), and extracted with ethyl acetate (20 mL×2). The combined organic phase was washed with brine (10 mL), dried with anhydrous Na₂SO₄, filtered and concentrated in vacuum to afford the title compound (180 mg, 425.97 μmol, 62.0% yield) as a colorless oil.

LCMS: m/z 423.1 [M+H]+.

Step 6: 1-methylpyrrolidin-3-yl 3-(3-(3,5-dimethyl-1-((2-(trimethylsilyl)ethoxy)-methyl)-1H-pyrazol-4-yl)propoxy)-4-fluorobenzoate

To a solution of 3-(3-(3,5-dimethyl-1-((2-(trimethylsilyl)ethoxy)methyl)-1H-pyrazol-4-yl)propoxy)-4-fluorobenzoic acid (160 mg, 378.64 mol) in DMF (3 mL) was added CDI (92.09 mg, 567.96 mol). The mixture was stirred at 25° C. for 0.5 h. Then the mixture cooled to 5° C. was added 1-methylpyrrolidin-3-ol (57.45 mg, 567.96 μmol) and NaH (30.29 mg, 757.28 μmol, 60% purity). The mixture was stirred at 60° C. for 12 h. The mixture was quenched with NH₄Cl solution (20 mL). The aqueous phase was extracted with EtOAc (20 mL×2). The combined organic phase was washed with brine (10 mL), dried with anhydrous Na₂SO₄, filtered and concentrated in vacuum. The residue was purified by prep-TLC (SiO₂, DCM/MeOH=10/1) to afford the title compound (60 mg, 118.65 μmol, 31.3% yield) as a colorless oil.

LCMS: m/z 506.4 [M+H]+.

Step 7: 1-methylpyrrolidin-3-yl 3-(3-(3,5-dimethyl-1H-pyrazol-4-yl)propoxy)-4-fluorobenzoate

To a solution of 1-methylpyrrolidin-3-yl3-(3-(3,5-dimethyl-1-((2-(trimethylsilyl)ethoxy)methyl)-1H-pyrazol-4-yl)propoxy)-4-fluorobenzoate (55 mg, 108.76 μmol) in DCM (1 mL) was added TFA (770.00 mg, 6.75 mmol, 0.5 mL). The mixture was stirred at 25° C. for 12 h. The mixture was poured into NH₄Cl solution (40 mL). The aqueous phase was extracted with DCM (20 mL×2). The combined organic phase was washed with brine (10 mL), dried with Na₂SO₄, filtered and concentrated in vacuum. The residue was purified by prep-HPLC (column: Phenomenex Gemini-NX C18 75×30 mm×3 μm; mobile phase: (water (10 mM NH₄HCO₃)-ACN); B %: 27%-57%, 7 min) to afford the title compound (15.25 mg, 40.42 μmol, 37.2% yield, 99.5% purity) as an off-white gum.

LCMS: m/z 376.1 [M+H]+.

¹H NMR (400 MHz, CDCl₃) δ=7.64-7.62 (m, 1H), 7.43 (dd, J=2.0, 8.0 Hz, 1H), 7.10 (dd, J=8.4, 10.8 Hz, 1H), 5.44 (br s, 1H), 4.02 (t, J=6.4 Hz, 2H), 3.11-2.92 (m, 2H), 2.85-2.70 (m, 1H), 2.58 (t, J=7.2 Hz, 2H), 2.53-2.40 (m, 5H), 2.18 (s, 6H), 2.00-1.94 (m, 3H).

### Example 3

A stability assay in plasma (rat or human) is used to evaluate the ability of a compound provided herein to convert to an active TTR stabilizer. The test compound is added to plasma and incubated at 37° C. in a water bath at a concentration of 2 μM. At each time point (0, 10, 30, 60 and 120 min or 0, 60, 120, 180 and 240 min), stop solution (tolbutamide plus labetalol) is added to precipitate protein and mixed thoroughly. After centrifugation, an aliquot of supernatant is analyzed by LC-MS/MS. The percentage of formation of active agent is calculated at each time point.

### Example 4

A stability assay in liver S9 (rat or human) was used to evaluate the ability of a compound provided herein to convert to an active TTR stabilizer. The test compound was added to liver S9 and incubated at 37° C. in a water bath at a concentration of 1 μM. At each time point (0, 5, 10, 20, 30 and 60 min), stop solution (tolbutamide plus labetalol) was added to precipitate protein and mixed thoroughly. After centrifugation, an aliquot of supernatant was analyzed by LC-MS/MS. The percentage of formation of active agent was calculated at each time point.

### Example 5

For a compound provided herein to be an effective TTR stabilizer drug to halt and/or prevent the ocular and cerebral TTR amyloid deposition TTR amyloidosis, it has to be able to penetrate into the brain and CSF (surrogate for eye penetration) and deliver a sufficient amount of TTR stabilizer to stop TTR dissociation. A pharmacokinetic study in rat is used to evaluate the compounds. Male Sprague-Dawley (SD) rats (200-220 g weight) are acclimated for at least 2 to 3 days before being placed on study. All animals will have access to certified rodent diet and water at libitum. Appropriate amount of the test compound is accurately weighed and mixed with appropriate volume of vehicle (such as DMSO/sterile water for iv dosing or 0.5% methylcellulose homogenous suspension or solution for oral administration or as a solution in a mixture NMP/PEG400/solutol/water) to administer a dose of 2, 5 or 10 mg/kg. For IV dosing, the test compound is administered via tail vein or indwelling cannula. For oral dosing, the test compound is administered by oral gavage. Blood and CSF samples are collected at selected timepoints. Blood collection is performed from saphenous vein or tail vein of each animal into polypropylene tubes at each timepoint. All blood samples are transferred into EDTA-K2 tubes and centrifuged for 15 minutes at 4° C. for plasma collection. Plasma samples are kept at −80° C. until LC/MSMS analysis. CSF is collected from cisterna magna at each timepoint and quick frozen over dry ice and kept at −80° C. until LC/MSMS analysis. Brains are harvested immediately after the terminal bleeding (˜24 hrs post dosing). The blood of the brain is perfused with normal saline. The brain is quickly picked and placed into centrifuge tube. The weight of brain samples is recorded. 4-Fold homogenization solution (MeOH/15 mM PBS (1:2)) is added into the tube according to the weighed samples. The brain is homogenized using a Polytron (3 strokes or more until homogenous, each 30 seconds) on wet ice. The samples are quick frozen over dry ice and kept at −80° C. until LC/MSMS analysis. Using a LC-MSMS method for the quantitative determination of test compound in biological matrixes, amount of test compound and active agent are measured in plasma and CSF at selected timepoints post-dosing and in brain at 24 hrs post dosing. Plasma concentration versus time data is analyzed by non-compartmental approaches using the Phoenix WinNonlin 6.3 software program. As reference, an oral dose of 40 mg/kg of AG10 gives a CSF to plasma ratio over 24 hrs around 0.009 and a brain to plasma ratio at 24 hrs around 0.008.

### Results

Results from the rat liver S9 assays are shown in Table 2.

TABLE 2
In vitro Rat Liver S9 Stability Assay -
Formation of AG10 at 60 min

| Compound ID | Formation* |
|---|---|
| 1 | A |
| 2 | A |

*Formation: A is <25%, B is ≥25 to <50%, C is ≥50 to <75% and D is ≥75%

This disclosure is not to be limited in scope by the embodiments disclosed in the examples which are intended as single illustrations of individual aspects, and any equivalents are within the scope of this disclosure. Various modifications in addition to those shown and described herein will become apparent to those skilled in the art from the foregoing description. Such modifications are intended to fall within the scope of the appended claims.

Various references such as patents, patent applications, and publications are cited herein, the disclosures of which are hereby incorporated by reference herein in their entireties.

What is claimed is:

1. A compound having the structure of Formula (I): 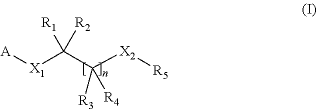
or a pharmaceutically acceptable salt thereof, wherein:
A is 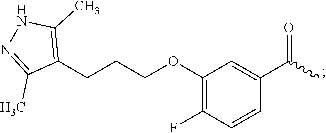
X₁ is O, S or NR₆;
X₂ is a bond, O, S, NR₆, N⁺R₆R₇ or P⁺(Ar)₂;
X₃ is O, S or NR₆;
n is an integer from 0-6;
m is an integer from 0-6;
Ar is aryl, heteroaryl or heteroarylium, all optionally substituted;
R₁, R₂, R₃, R₄, R₅, R₆ and R₇ are each independently H, alkyl, haloalkyl, cycloalkyl, heterocycloalkyl, alkenyl, cycloalkenyl, heterocycloalkenyl, aryl, heteroaryl, aralkyl or heteroaralkyl, all optionally substituted, or OH, OR₈, COR₉, —(CR₁₀R₁₁)ₘX₃R₈ or —(CR₁₀R₁₁)ₘX₃COR₉;
R₈ and R₉ are each independently H, alkyl, haloalkyl, cycloalkyl, heterocycloalkyl, alkenyl, cycloalkenyl, heterocycloalkenyl, aryl, heteroaryl, aralkyl or heteroaralkyl, all optionally substituted;
or R₈ is selected as above and R₉ is 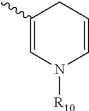
R₁₀ and R₁₁ are each independently H, alkyl, haloalkyl, cycloalkyl, heterocycloalkyl, alkenyl, cycloalkenyl, heterocycloalkenyl, aryl, heteroaryl, aralkyl or heteroaralkyl, all optionally substituted, or OH or OR₈;
or R₁-R₂ form a cycloalkyl or heterocycloalkyl; and R₃-R₁₁ are selected as above;
or R₃-R₄ form a cycloalkyl or heterocycloalkyl; and R₁, R₂ and R₅-R₁₁ are selected as above;
or R₁₀-R₁₁ form a cycloalkyl or heterocycloalkyl; and R₁-R₉ are selected as above;
or R₁-R₃ form a bond, —(CR₁₀R₁₁)ₘ— or —(CR₁₀═CR₁₁)ₘ—; and R₂ and R₄-R₁₁ are selected as above;
or R₂-R₄ form a bond, —(CR₁₀R₁₁)ₘ— or —(CR₁₀═CR₁₁)ₘ—; and R₁, R₃ and R₅-R₁₁ are selected as above;
or R₁-R₅ form a bond, —(CR₁₀R₁₁)ₘ— or —(CR₁₀═CR₁₁)ₘ—; and R₂ -R₄ and R₆-R₁₁ are selected as above;
or R₂-R₆ form a bond, —(CR₁₀R₁₁)ₘ— or —(CR₁₀═CR₁₁)ₘ—; and R₁, R₃-R₅ and R₇-R₁₁ are selected as above;
or R₃-R₅ form a bond, —(CR₁₀R₁₁)ₘ—, —(CR₁₀═CR₁₁)ₘ— or —[C(R₁₀)═C(R₁₁)—CO]—; and R₁, R₂, R₄ and R₆-R₁₁ are selected as above;
or R₄-R₆ form a bond, —(CR₁₀R₁₁)ₘ— or —(CR₁₀═CR₁₁)ₘ—; and R₁-R₃, R₅ and R₇-R₁₁ are selected as above;
or R₅-R₆ form —(CR₁₀R₁₁)ₘ— or —(CR₁₀R₁₁)ₘ—X₃—(CR₁₀R₁₁)ₘ—; and R₁-R₄ and R₇-R₁₁ are selected as above;
with the provisos that:
when X₁ is oxygen, X₂ is a bond, and R₁, R₂ and R₅ are H, then n is not 0; and
when X₁ is NH, X₂ is a bond, and R₁, R₂, R₃, R₄ and R₅ are H, then n is not 1.

2. A compound having the structure of Formula (II): 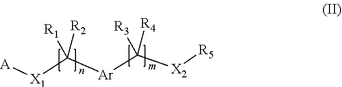
or a pharmaceutically acceptable salt thereof, wherein:
A is 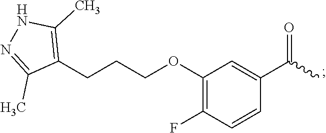
X₁ O, S or NR₆;
X₂ is a bond, O, S, NR₆, N⁺R₆R₇ or P⁺(Ar)₂;
X₃ is O, S or NR₆;
n is an integer from 0-6;
m is an integer from 0-6;
Ar is aryl, heteroaryl or heteroarylium, all optionally substituted;
R₁, R₂, R₃, R₄, R₅, R₆ and R₇ are each independently H, alkyl, haloalkyl, cycloalkyl, heterocycloalkyl, alkenyl, cycloalkenyl, heterocycloalkenyl, aryl, heteroaryl, aralkyl or heteroaralkyl, all optionally substituted, or OH, OR₈, COR₉, —(CR₁₀R₁₁)ₘX₃R₈ or —(CR₁₀R₁₁)ₘX₃COR₉;
R₈ and R₉ are each independently H, alkyl, haloalkyl, cycloalkyl, heterocycloalkyl, alkenyl, cycloalkenyl, heterocycloalkenyl, aryl, heteroaryl, aralkyl or heteroaralkyl, all optionally substituted;
or R₈ is selected as above and R₉ is 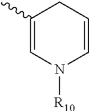
R₁₀ and R₁₁ are each independently H, alkyl, haloalkyl, cycloalkyl, heterocycloalkyl, alkenyl, cycloalkenyl, heterocycloalkenyl, aryl, heteroaryl, aralkyl or heteroaralkyl, all optionally substituted, or OH or OR₈;
or R₁-R₂ form a cycloalkyl or heterocycloalkyl; and R₃-R₁₁ are selected as above;
or R₃-R₄ form a cycloalkyl or heterocycloalkyl; and R₁, R₂ and R₅-R₁₁ are selected as above;
or R₁₀-R₁₁ form a cycloalkyl or heterocycloalkyl; and R₁-R₉ are selected as above;
or R₁-R₃ form a bond, —(CR₁₀R₁₁)ₘ— or —(CR₁₀═CR₁₁)ₘ—; and R₂ and R₄-R₁₁ are selected as above;
or R₂-R₄ form a bond, —(CR₁₀R₁₁)ₘ— or —(CR₁₀═CR₁₁)ₘ—; and R₁, R₃ and R₅-R₁₁ are selected as above;
or R₁-R₅ form a bond, —(CR₁₀R₁₁)ₘ— or —(CR₁₀═CR₁₁)ₘ—; and R₂-R₄ and R₆-R₁₁ are selected as above;
or R₂-R₆ form a bond, —(CR₁₀R₁₁)ₘ— or —(CR₁₀═CR₁₁)ₘ—; and R₁, R₃-R₅ and R₇-R₁₁ are selected as above;
or R₃-R₅ form a bond, —(CR₁₀R₁₁)ₘ—, —(CR₁₀═CR₁₁)ₘ— or —[C(R₁₀)═C(R₁₁)—CO]—; and R₁, R₂, R₄ and R₆-R₁₁ are selected as above;
or R₄-R₆ form a bond, —(CR₁₀R₁₁)ₘ— or —(CR₁₀═CR₁₁)ₘ—; and R₁-R₃, R₅ and R₇-R₁₁ are selected as above;
or R₅-R₆ form —(CR₁₀R₁₁)ₘ— or —(CR₁₀R₁₁)ₘ—X₃—(CR₁₀R₁₁)ₘ—; and R₁-R₄ and R₇-R₁₁ are selected as above.

3. The compound having one of the following structures:

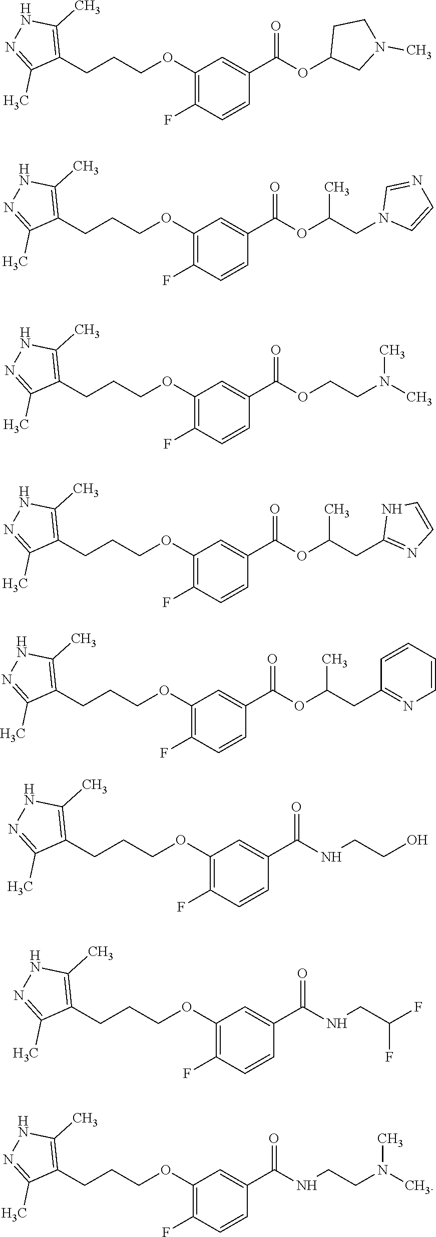

4. A pharmaceutical composition, comprising the compound of claim 1, 2 or 3, and a pharmaceutically acceptable excipent.

5. A method of inhibiting and preventing TTR aggregation and/or amyloid formation in the eye or CNS of a subject, comprising administering to the subject the compound of claim 1, 2 or 3.

6. A method of treating a subject having peripheral TTR amyloidosis or ocular or cerebral amyloid angiopathy, comprising administering to the subject the compound of claim 1, 2 or 3.

7. A method of treating a subject having familial amyloid polyneuropathy, familial amyloid cardiomyopathy, localized nodular cutaneous amyloidosis, TTR oculoleptomeningeal amyloidosis or senile systemic amyloidosis, comprising administering to the subject the compound of claim 1, 2 or 3.
